(19) United States
(12) Patent Application Publication
(10) Pub. No.: US 2007/0072814 A1
(43) Pub. Date: Mar. 29, 2007

(54) Method and means of modulating lipid metabolism

(76) Inventor: Ivan Petyaev, Cambridgeshire (GB)

Correspondence Address: NIXON & VANDERHYE, PC, 901 NORTH GLEBE ROAD, 11TH FLOOR, ARLINGTON, VA, 22203, US

(21) Appl. No.: 10/574,852

(22) PCT Filed: Sep. 29, 2004
(86) PCT No.: PCT/GB04/04162

§ 371 Date: Oct. 19, 2006

(30) Foreign Application Priority Data
Oct. 6, 2003 (GB) 0323348.3

(51) Int. Cl. A61K 31/7052; A61K 31/60

(52) U.S. Cl. 514/28; 514/165

(57) ABSTRACT

The present invention relates to modulation of lipid metabolism, in particular reductions in the levels of total cholesterol and apo-lipoprotein, of an individual. This modulation is achieved by combined administration of both anti-microbial and metal-chelator compounds. Various therapeutic applications of this lipid modulation are provided.

The present invention relates to modulation of lipid metabolism within the vascular system of an individual.

Abnormalities in the transport and metabolism of lipids within the vascular system are associated with hyperlipidaemia and other medical conditions. Hyperlipidaemias are the primary metabolic disease in the developed world and are associated with a range of conditions, including diabetes, obesity, cardio-vascular pathology, renal failure, nephrotic syndrome, alcohol abuse, cirrhosis of the liver and hypothyroidism (Durrington, P. N. Hyperlipidaemia: diagnosis and management. Wright, London, 1989; Havel, R. J. and Rapaport, E. New England Journal of Medecine, 1995, 332, 1491-1498).

Hyperlipidaemia and other abnormalities in lipid metabolism may be identified by measuring levels of one or more serum markers such as total cholesterol, LDL-cholesterol, apolipoprotein B and triglycerides. Aberrant levels of one or more of these markers in an individual are characteristic of hyperlipidaemia and other medical conditions.

Current lipid regulating drugs are ineffective in certain groups of patients and new agents for the selective reduction of the levels of these markers in the vascular may be useful in promoting health and reducing the risk of cardiovascular disease and other medical conditions.

The present inventor has now discovered that anti-microbial and metal-chelator compounds, when administered together, have an unexpected effect on lipid metabolism, in particular reducing levels of total cholesterol levels and apo-lipoprotein B. This effect is not observed using these compounds alone.

A first aspect of the present invention provides the use of an anti-microbial compound and a metal chelator in the manufacture of a medicament for modulating lipid metabolism in the vascular system of an individual.

An anti-microbial compound may be any compound that is active in preventing, reducing or ameliorating microbial infection. Suitable anti-microbial compounds include tetracyclin, ofloxacin, clinafloxacin, ciprofloxacin, clindamycin, doxycycline and minocycline. Preferred anti-microbial compounds include macrolide antibiotics such as erythromycin or azalides such as trythromycin, roxithromycin, zithromycin, clarithromycin and azithromycin.

In some embodiments, a suitable anti-microbial compound may be a low pH anti-oxidant compound i.e. a compound which has antioxidant activity at pH 5-6. Examples of low pH anti-oxidant compounds include azithromycin. Anti-oxidant activity may be determined as described in the experimental section below.

A metal chelator may include desferrioxamine mesylate, haem derivatives, penicillamine, tiopronin, trientine, dihydrochloride, diethyldithiocarbamate, acetylsalicylic acid, disodium/trisodium, edetate, edetic acid and unithiol. In particular, copper chelators such as penicillamine, tiopronin, trientine, dihydrochloride, diethyldithiocarbamate and acetylsalicylic acid may be used.

An individual may be suffering from a disorder of lipid metabolism, such as hypercholesterolemia, hyperlipidemia, nephrotic syndrome, hypothyroidism, dysglobulinemia or Cushing syndrome. Such an individual may have elevated levels of apo-B and/or total cholesterol in the bloodstream in relation to the population as a whole

Alternatively, an individual may not be suffering from a disorder of lipid metabolism and may have levels of cholesterol or apo-B in the bloodstream which fall within the normal range i.e. are not elevated in relation to the population as a whole. Reduction of cholesterol and apo-B levels may still be desirable in these individuals to promote health and reduce susceptibility to disease.

In preferred embodiments, lipid metabolism may be modulated in the vascular system of an individual who is not suffering from an atherosclerotic condition. Such an individual may show none of the characteristic features of an atherosclerotic condition, such as narrowed arteries, ECT irregularities and/or an abnormal ankle/branchial index.

Modulation of lipid metabolism may include reducing total cholesterol levels and/or reducing Apo-B levels. Total cholesterol is total amount of cholesterol carried in the blood by LDL, HDL and other carriers. Elevated levels of total cholesterol, for example >200mg/dL or >240mg/dL, may be indicative of an increased risk of suffering from a medical condition, such as cardiovascular disease. Apolipoprotein B is the predominant protein component of Low density lipoproteins (LDL) and plays an important role in directing the formation and metabolism of LDL, which are major carriers of plasma cholesterol in man. The reduction of apo-B levels as described herein without a concomitant decrease in LDL-cholesterol may be of therapeutic benefit in reduces the metabolic impact of LDL-cholesterol.

Their role is to transport cholesterol to tissues where it may be needed for membrane structure or conversion into various metabolites such as steroid hormones. Combinations of anti-microbial agents and metal chelators as described above may be used simultaneously or sequentially to affect lipid metabolism in the vascular system of an individual. The precise choice of agents, doses, duration and other parameters may be determined according to the individual case by a medical practitioner. This efficacy of a particular treatment may be determined for each individual case by monitoring changes in LDL levels in the serum of the treated patients using methods described herein

The anti-microbial compound and metal chelator may be administered sequentially or concomitantly to the individual.

Other aspects of the present invention provides the use of an anti-microbial compound in the manufacture of a medicament for use in combination with a metal chelator in modulating the lipid metabolism in the vascular system of an individual and the use of metal chelator in the manufacture of a medicament for use in combination with a anti-microbial compound in modulating the lipid metabolism in the vascular system of an individual.

Another aspect of the invention provides a method for modulation lipid metabolism in the vascular system comprising administering an anti-microbial compound and a metal chelator sequentially or concomitantly to an individual in need thereof.

Anti-microbial compounds and metal chelators are described in detail above.

A method may comprise determining the level of apo-B and/or cholesterol in a sample obtained from the individual before, during and/or after said treatment, for example a blood, plasma or serum sample.

As described above, the individual may have aberrant lipid metabolism and may, for example, be suffering from a disorder of lipid metabolism.

Another aspect of the invention provides a therapeutic system comprising an anti-microbial compound and a metal chelator for modulation of lipid metabolism in the vascular system of an individual.

An anti-microbial compound and a metal chelator may be administered in the form of a pharmaceutical composition. A composition may include, in addition to the above agents, a pharmaceutically acceptable excipient, carrier, buffer, stabiliser or other materials well-known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

The invention thus provides a pharmaceutical composition comprising an anti-microbial agent, a metal chelator and a pharmaceutically acceptable excipient for use in the modulation of lipid metabolism, as described herein.

It will be appreciated that appropriate dosages of the anti-microbial and metal chelator compounds and compositions comprising these compounds, can vary from patient to patient. Determining the optimal dosage will generally involve the balancing of the level of therapeutic benefit against any risk or deleterious side-effects.

The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations within the brain which achieve the desired effect. Further details of appropriate dosages are found in the British National Formulary (2000) Pub: British Medical Association & Royal Pharmacological Society of Great Britain.

Administration in vivo can be effected in one dose, continuously or more preferably, intermittently, for example at regular intervals throughout the course of treatment. Methods of determining the most effective means and dosage of administration are well-known to those of skill in the art and will vary with the formulation used for therapy and the subject being treated. Single or multiple administrations can be carried out with the dose level and pattern being selected by the treating physician.

The anti-microbial agent and metal chelator or composition comprising these compounds may be administered to a subject by any convenient route of administration.

Routes of administration include, but are not limited to, oral, for example by ingestion, and parenteral, for example, by cutaneous, subcutaneous or intravenous injection; or by implant of a depot or reservoir, for example, subcutaneously or intramuscularly.

Compositions of the present invention may conveniently be formulated in unit dosage form and may be prepared by any methods well known in the art of pharmacy. Formulations may, for example, be in the form of liquids, solutions, suspensions, emulsions, tablets, capsules, cachets, pills or ampoules.

For parenteral administration (e.g., by injection, including cutaneous, subcutaneous, intramuscular, intravenous and intradermal), the active ingredients will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

Pharmaceutical compositions for oral administration (i.e. by ingestion) may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

A tablet may be made by compression or moulding, optionally with one or more accessory ingredients. Compressed tablets may be prepared by compressing in a suitable machine the active ingredient in a free-flowing form such as a powder or granules, optionally mixed with other ingredients. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Other aspects of the invention relate to the use of an low pH anti-oxidant compound and a metal chelator in the manufacture of a medicament for modulating lipid metabolism in the vascular system of an individual and a method for modulating lipid metabolism in the vascular system comprising administering a low pH anti-oxidant compound and a metal chelator to an individual in need thereof.

A pharmaceutical composition for use in accordance with these aspects of the invention may comprise a low pH anti-oxidant compound, a metal chelator and a pharmaceutically acceptable excipient. The composition may be suitable for use in the modulation of lipid metabolism, as described above. The formulation of pharmaceutical compositions is described in more detail above.

Preferred low pH anti-oxidant compounds have anti-oxidant activity at pH 5-6 and include macrolide compounds and azalides such as azithromycin. Antioxidant activity may be determined as described below. Preferred metal chelators are described in more detail above.

Modulation of lipid metabolism may include reducing total cholesterol levels and/or reducing Apo-B levels as described above.

Various further aspects and embodiments of the present invention will be apparent to those skilled in the art in view of the present disclosure. All documents referenced in this specification are incorporated herein by reference.

All combinations and sub-combinations of the features described above are encompassed by the invention. Certain aspects and embodiments of the invention will now be illustrated by way of example and with reference to the table described below.

Table 1 shows the effect of anti-microbial treatment on the level of LDL in the serum of human blood.

Table 2 shows examples of anti-microbial agents.

Table 3 shows the clinical data indicating the effect to of treatment as described herein.

Experimental

Materials and Methods

Measurement of Antioxidant Activity at Different pH.

Aliquots of human serum were mixed with equal volumes of different buffers in order to achieve a range of samples with different final pH values, as described below.
1. To 1.0 ml of human serum, pH 7.4, 1.0 ml of 0.14 M acetate buffer pH 3.8 was added. As a result of this a sample of 2.0 ml of diluted serum with pH 5.6 was obtained.
2. 10 μl of a testing compound, at a chosen concentration, should be added to 1.0 ml of this diluted serum.
3. To another 1.0 ml of this serum, control sample, 10 μl of 0.14M 5.6 acetate buffer should be added.
4. To initiate peroxidation of lipoproteins in both serum samples 1 μg of atheroma IgG in volume of 10 μl should be added to each of them.
5. After that these samples should be incubated at 37° C. overnight and the level of accumulated malondialdehyde, product of lipid peroxidation, should be measured.
6. A difference in the concentration of this product between the control sample and the sample where the testing compound is present is a measure of antioxidant activity of the testing compound.

### CLINICAL EXAMPLES

A group of 35 patients were selected for therapy to alter lipid metabolism relative to a control group of 20 ‘matched’patients who were not treated (Patient Control Group).

The therapy group comprised 23 male and 7 female patients with an average age of 55±1.1 years. The patient control group was comprised of 20 patients with IHD, of which 15 were male and 5 were female with an average age of 53±1.2 years. Each patient gave written consent for his/her participation in the trial.

All patients had angina of II-III class of Canadian Cardiological Society classification. 15 patients in the therapy group and 10 in the patient control group had a history of myocardial infarction in the past year. IHD diagnosis for the other 15 patients with a recent history of unstable angina in the first group and 10 in the patient control group was confirmed by coronary angiography, which detected 70% or more of arterial stenosis.

Apart from the degree of generalization or severity of atherosclerosis, all groups were matched not only in age, gender and risk factors but also in medication, nitrates, β-blockers, angiotensin-converting enzyme inhibitors etc.

Progression of the clinical condition of the patients was monitored by the use of the modified Bruce Protocol for treadmill exercise/stress ECG testing and on the Rose-Blackburn Questionnaire (Cardiovascular Survey Methods. WHO, Geneva, 1968).

The therapy group was split into 4 therapeutic sub-groups:
1.) Therapy group A, 11 patients, —given azithromycin in a dose of 500 mg daily.
2.) Therapy group B, 8 patients, —a combined administration of azithromycin, in the same dose, with acetylsalicylic acid (aspirin) was prescribed. The dose of aspirin was 250 mg per day.
3.) Therapy Group C, 9 patients, —a combined administration of azithromycin, in the same dose as in the previous groups, and vitamins E, A, C, was prescribed. The daily dose of vitamin E was 30 mg, vitamin A 1,500 EU and vitamin C 90 mg.
4.) Therapy Group D, 7 patients, —The patients in this group were given 250 mgs aspirin daily only.

Each therapy was administered for 8 weeks. The blood of the patients of all four groups was tested every two weeks.

The severity of the clinical condition of the patients was estimated by using a modified Rose G., Blackburn H. Questionnaire.

The level of LDL was estimated in two independent parameters: LDL-cholesterol measured by combination of enzymatic and immunologic assays, and Apo-B measured by immuno-turbometric assay. These assays were performed using commercially available kits which include goat anti-human apo-B polyclonals (LDL-Direct™, Randox Labs Ltd UK, EZ LDL™ Cat No-358-A, Sigma UK).

The most significant effect on the serum parameters of lipid metabolism, more than 30% change in them from their initial concentration, was observed in Group B, where a combination of azithromycin and aspirin was used. In this group reduction in total cholesterol was 49%. This effect continued to be observed 2 months after treatment. The use of a combination of azithromycin with antioxidants was less effective. The use of either azithromycin, or aspirin, or antioxidants alone had no cholesterol lowering effect.

Changes in low density lipoproteins (LDL) were also observed in the patient sera. These changes were significant only for Group B where a combination of azithromycin and aspirin was used. As result of this therapy, the protein component of these lipoproteins, ApoB, was reduced by 38%, whilst their lipid content, in terms of cholesterol, was not significantly affected. This indicates that the therapy, apart from its suppression of the total cholesterol concentration, may also specifically target synthesis or metabolism of the protein part of LDL, ApoB.

TABLE 1

| Antibacterial | |
|---|---|
| agents | Proprietary Preparations (all trademarks) |
| Tetracycline | Aust: Achromycin; Actisite; Hostacyclin; Latycin; |
| | Steclin; tetrarco; Austral.: Achromycin; Achromycin |
| | V; Latycin; Mysteclin; Panmycin P; Steclin-V; |
| | Tetramykoin; Tetrex; Belg.: Hostacucline; Canad.: |
| | Achromycin; Achromycin V; Apo-Tetra; Novo-Tetra; |
| | Nu-Tetra; Tetracyn; Fr.: Florocycline; Hexacycline; |
| | Tetramig; Ger.: Achromycin; Akne-Pyodron Kur; Akne- |
| | Pyodron oral; Dispatetrin; Hostacyclin; Imex; |
| | Quimocyclin N; Sagittacin N; Steclin; Supramycin; |
| | Tefilin; Tetrabakat; Tetrablet; Tetracitro S; |
| | Tetralution; Ital.: Acromicina; Ambramicina; |
| | Calociclina; Ibicyn; Spaciclina; Tetra-Proter; |
| | Tetrabioptal; Tetrafosammina; Neth.: Tetrarco; S.Afr.: |
| | Achromycin; Arcanacycline; Gammatet; Hostacycline; |
| | Rotet; Tetrex; Spain: Actisite; Ambramicia; |
| | Britaciclina; Kinciclina; Quimpe Antibiotico; Tetra |
| | Hubber; Tetralen; Tetrarco Simple; Swed.: Achromycin; |
| | Actisite; Switz.: Achromycine; Actisite; Servitet; |
| | Tetraseptine; Triphacycline; UK: Achromycin; |
| | Economycin; Sustamycin; Tetrabid-Organon; Tetrachel; |
| | USA: Achromycin V; Achromycin; Actisite; Nor-Tet; |
| | Panmycin; Robitet Robicaps; Sumycin; Teline; Tetracap; |
| | Tetralan; Tetram.* |
| Erythromycin | |
| Azithromycin | |
| Roxithromycin | |
| Ofloxacin | |
| Clinafloxacin | |
| Ciprofloxacin | |
| Clindamycin | |
| Doxycycline | |
| Minocycline | |

TABLE 2

| Metals | Chelators | Proprietary Preparations (all trademarks) |
|---|---|---|
| Fe⁺²/ | Desferri- | Canad.: Zinecard; Fr.: Cardioxane; Ital.: |
| Fe⁺³ | oxamine | Cardioxane; Eucardion; USA: Zinecard. |
| | Mesylate | |
| | Haem | Austral.: Panhematin; Fr.: Normosang; |
| | Derivatives | USA: Panhematin. |
| Cu⁺¹/ | Penicil- | Aust.: Artamin; Distamine; Austral.: |
| Cu⁺² | lamine | D-Penamine; Belg.: Kelatin; Canad.: |
| | | Cuprimine; Depen; Fr.: Trolovol; Ger.: |
| | | Metacaptase; Trisorcin; Trolovol; Irl.: |
| | | Distamine; Ital.: Pemine; Sufortan; Neth.: |
| | | Cuprimine, Distamine; Gerodyl; Kelatin; |
| | | Norw.: Cuprimine; S.Afr.: Metaalcaptase; |
| | | Spain: Cuprein; Sufortanon; Swed.: |
| | | Cuprimine; Switz.: Mercaptyl; UK: |
| | | Distamine, Pendramine; USA: Cuprimine; |
| | | Depen. |
| | Tiopronin | Fr.: Acadione; Ger.: Captimer; Ital.: |
| | | Epatiol; Mucolysin; Mucosyt; Thiola; |
| | | Tioglis; Spain: Sutilan; Switz.: Mucolysin; |
| | | USA: Thiola. Multi-ingredient: Ital.: |
| | | Mucolysin Antibiotico; Spain: Hepadigest. |
| | Trientine | USA: Syprine. |
| | Dihydro- | |
| | chloride | |
| | Diethyldi- | |
| | thiocarbamate | |
| | Acetylsali- | |
| | cylic acid | |
| Me⁺²* | Disodium/ | Fr.: Chelatran; Tracemate; Irl.: Limclair; |
| | Trisodium | UK: Limclair; USA: Disotate; Endrate. |
| | Edetate | Multi-ingredient: Canad.: Murine |
| | | Supplement Tears; Fr.: Vitaclair; Ger.: |
| | | Complete; Duracare; Oxysept; UK: Uriflex |
| | | G; Uriflex R. |
| | Edetic Acid | Multi-ingredient: Ital.: Conta-Lens |
| | | Wetting; USA: Summer'sEve Post-Menstrual; |
| | | Triv; Vagisec Plus; Zonite. |
| | Unithiol | Ger.: Dimaval; Mercuval. |

*Any bivalent metal

TABLE 3

| Group | Triglycerides | Total cholesterol | LDL cholesterol | ApoB | ApoA |
|---|---|---|---|---|---|
| Before | | | | | |
| treatment | | | | | |
| Group A | 117 | 205 | 34 | 125 | 161 |
| Group B | 115 | 285 | 37 | 119 | 153 |
| Group C | 109 | 259 | 37 | 120 | 156 |
| Group D | 116 | 253 | 37 | 124 | 158 |
| | 114 | 251 | 36 | 122 | 157 |
| Treatment | | | | | |
| 2 weeks | | | | | |
| Group A | — | — | — | — | — |
| Group B | — | — | — | — | — |
| Group C | 98 (85%) | 197 (76%) | 38 (103%) | 119 (99%) | 150 (96%) |
| Group D | 152 (131%) | 233 (92%) | 41 (110%) | 137 (110%) | 157 (99%) |
| Treatment | | | | | |
| 4 weeks | | | | | |
| Group A | 112 (96%) | 186 (91%) | 37 (109%) | 109 (87%) | 142 (88%) |
| Group B | 94 (82%) | 171 (60%) | 44 (119%) | 77 (65%) | 124 (81%) |
| Group C | 87 (80%) | 184 (71%) | 41 (111%) | 102 (85%) | 144 (92%) |
| Group D | 111 (96%) | 210 (83%) | 41 (111%) | 113 (91%) | 151 (96%) |
| Treatment | | | | | |
| 6 weeks | | | | | |
| Group A | 101 (86%) | 190 (93%) | 38 (112%) | 110 (88%) | 143 (89%) |
| Group B | 98 (85%) | 153 (53%) | 46 (124%) | 77 (65%) | 123 (80%) |
| Group C | 96 (88%) | 190 (73%) | 40 (108%) | 116 (97%) | 149 (96%) |
| Group D | — | — | — | — | — |
| Treatment | | | | | |
| 8 weeks | | | | | |
| Group A | 99 (85%) | 191 (93%) | 39 (115%) | 113 (90%) | 141 (88%) |
| Group B | 89 (77%) | 145 (51%) | 44 (119%) | 74 (62%) | 115 (75%) |
| Group C | — | — | — | — | — |
| Group D | — | — | — | — | — |
| 1 month after | | | | | |
| treatment | | | | | |
| Group A | 106 (91%) | 197 (96%) | 44 (129%) | 115 (92%) | 145 (90%) |
| Group B | 109 (95%) | 174 (61%) | 43 (116%) | 114 (96%) | 146 (95%) |
| Group C | 122 (112%) | 201 (78%) | 40 (108%) | 141 (117%) | 147 (94%) |
| Group D | 146 (126%) | 221 (87%) | 44 (119%) | 131 (106%) | 184 (116%) |
| 2 months after | | | | | |
| treatment | | | | | |
| Group A | 118 (101%) | 196 (96%) | 41 (121%) | 133 (106%) | 162 (101%) |
| Group B | 110 (96%) | 187 (66%) | 46 (124%) | 116 (97%) | 152 (99%) |
| Group C | 126 (116%) | 242 (93%) | 38 (103%) | 154 (128%) | 168 (108%) |
| Group D | 128 (110%) | 223 (88%) | 38 (103%) | 142 (114%) | 166 (106%) |
| 3 months after | | | | | |
| treatment | | | | | |
| Group A | 109 (93%) | 198 (97%) | 40 (118%) | 131 (105%) | 161 (100%) |
| Group B | 114 (99%) | 205 (72%) | 46 (124%) | 132 (111%) | 173 (111%) |
| Group C | 113 (104%) | 227 (88%) | 40 (108%) | 135 (112%) | 161 (103%) |
| Group D | 134 (116%) | 234 (92%) | 43 (116%) | 111 (90%) | 179 (113%) |

Concentration of all lipid parameters are in mg/dL.

1. Use of an anti-microbial agent and a metal chelator in the manufacture of a medicament for the modulation of lipid metabolism in the vascular system of an individual.

2. Use according to claim 1 wherein the level of cholesterol is reduced in the vascular system of the individual.

3. Use according to claim 1 wherein the level of apolipoprotein-B is reduced in the vascular system of the individual.

4. Use according to claim 3 wherein the condition is selected from the group consisting of hypercholesterolemia, hyperlipidemia, nephrotic syndrome, hypothyroidism, dysglobulinemia and Cushing syndrome.

5. Use according to claim 5 wherein said anti-microbial agent is a macrolide antibiotic.

6. Use according to claim 5 wherein said anti-microbial agent is a azalide antibiotic.

7. Use according to claim 6 wherein said anti-microbial agent is azithromycin.

8. Use according to any one of the preceding claims claim 1 wherein said metal chelator is a copper chelator.

9. Use according to claim 8 wherein said copper chelator is acetylsalicylic acid.

10. Use according to claim 1 wherein said medicament is a single composition comprising an anti- microbial agent and a metal chelator.

11. Use according to claim 1 wherein said medicament comprises separate preparations of the anti- microbial agent and metal chelator.

12. A method for modulating lipid metabolism in the vascular system comprising administering an anti-microbial agent and a metal chelator to an individual in need thereof.

13. A method according to claim 12 wherein the total level of cholesterol is reduced in the vascular system of the individual.

14. A method according to claim 12 wherein the level of apolipoprotein-B is reduced in the vascular system of the individual.

15. A method according to claim 12 wherein the condition is selected from the group consisting of hypercholesterolemia, hyperlipidemia, nephrotic syndrome, hypothyroidism, dysglobulinemia and Cushing syndrome.

16. A method according to claim 12 wherein said anti-microbial agent is a macrolide antibiotic.

17. A method according to claim 16 wherein said anti- microbial agent is a azalide antibiotic.

18. A method according to claim 17 wherein said anti- microbial agent is azithromycin.

19. A method according to claim 12 wherein said metal chelator is a copper chelator.

20. A method according to claim 12 wherein said copper chelator is acetylsalicylic acid.

21. A method according to claim according to claim 12 wherein anti-microbial agent and the metal chelator are administered simultaneously.

22. A method according to claim 12 wherein anti-microbial agent and the metal chelator are administered sequentially.

23. A pharmaceutical composition comprising an anti-microbial agent and a metal-chelator for use in the modulation of lipid metabolism.

24. A pharmaceutical composition according to claim 23 comprising azithromycin and aspirin.

25. A method of preparing a composition for use in the modulation of lipid metabolism comprising; admixing a anti-microbial agent and a metal chelator with a pharmaceutically acceptable excipient.

26. A method according to claim 25 wherein the anti-microbial agent is azithromycin and the metal chelator is acetylsalicylic acid.
